(19) United States
(12) Patent Application Publication
(10) Pub. No.: US 2007/0269529 A1
(43) Pub. Date: Nov. 22, 2007

(54) Method For Treating Plants And Plant Parts

(76) Inventors: Keith Rowley, Madison, WI (US); Sang Won Jeong, Madison, WI (US); Keith Cowan, Stockholm (SE); James Altwies, Mazomanie, WI (US); Mark Trimmer, Madison, WI (US); Gurdip Brar, Middleton, WI (US); Mustafa Ozgen, Mersin (TR); Jiwan Palta, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/830,551

(22) Filed: Jul. 30, 2007

Related U.S. Application Data
(62) Division of application No. 10/750,083, filed on Dec. 31, 2003, now abandoned.
(60) Provisional application No. 60/438,016, filed on Jan. 3, 2003.
(60) Provisional application No. 60/486,275, filed on Jul. 10, 2003.

(51) Int. Cl. A61K 35/54

(52) U.S. Cl. 424/581

(57) ABSTRACT

Methods of using modified lecithin to delivery various benefits to plants and plant parts are disclosed. Modified lecithins, applied to growing plants, can cause improvements in fruit and plant firmness, size, color and stability, in economically important fruits and vegetables.

### CROSS-REFERENCE TO RELATED APPLICATIONS

This application is a divisional of U.S. application Ser. No.10/750,083, filed Dec. 31, 2003, which claims benefit to U.S. Provisional Application Ser. No. 60/438,016, filed on Jan. 3, 2003, and U.S. Provisional Application Ser. No. 60/486,275, filed on Jul. 10, 2003, all of which are hereby incorporated by reference herein.

### STATEMENT REGARDING FEDERALLY SPONSORED RESEARCH OR DEVELOPMENT

Not applicable.

### BACKGROUND OF THE INVENTION

Many plants and plant parts are of great economic importance to people. Fruit, vegetable, edible tuber and cut flower businesses are all multibillion dollar industries globally. Turf grass is another multibillion dollar industry. Over the years, people have learned to increase the production of various economically important plants and plant parts. Chemical agents have been applied to plants to increase the marketable yield of fruits, for example, by inhibiting fruit drop from the trees. In addition, people have learned to reduce the loss of economically important plant parts during the post-harvest storage and marketing period. In this regard, various chemical agents have been used to prolong the storage and shelf life of fruits, vegetables and cut flowers. However, many of the yield-increasing agents have the undesirable effect of causing the fruits and vegetables to soften and thus lead to poor storage and shelf life. Furthermore, many of the yield-increasing and the storage and shelf life-prolonging agents have toxicological and environmental concerns. There is a tremendous interest in the plant industry to find alternative agents.

Another major challenge to the plant industry relates to the protection of economically important plants from abiotic and biotic stress-related injuries. Specifically, over 60% of the crop loss in the U.S. from the late 1940s to the late 1990s was due to abiotic stresses (see USDA Agricultural Statistics, 1998). Abiotic stresses include chilling, freezing, drought, heat and other environmental factors. Biotic stresses, which include those caused by insects, nematodes, snails, mites, weeds, pathogens (e.g., fungus, bacteria and viruses), and physical damage caused by human and non-human animals, have also led to significant crop loss in the U.S. Thus, there is a tremendous interest in the plant industry to find a technology that can be used to prevent or mitigate stress injury and to accelerate recovery following a stress injury.

In the recent years, certain phospholipids such as lysophosphatidylethanolamine (LPE) have been found to be able to deliver some beneficial effects to various economically important plants and plant parts, which include protecting the plants from stress-related injuries (see WO 01/721330; and plant parts (see Farag, K. M. et al., Physiol. Plant, 87:515-524 (1993); Farag, K. M. et al., HortTech., 3:62-65 (1993); Kaur, N., et al., HortScience, 32:888-890 (1997); Ryu, S. B., et al., Proc. Natl. Acad. Sci. U.S.A., 94:12717-12721(1997); U.S. Pat. Nos. 5,126,155 and 5,110,341; and WO 99/23889). However, for large scale applications, these lysophospholipids are currently relatively expensive. Alternative agents that have the potential to provide cost effective delivery of the same or greater effects produced by the lysophospholipids are desired in the art.

### SUMMARY OF THE INVENTION

The present invention provides methods for delivering various beneficial effects to a plant or plant part by treating the plant or plant part with an effective amount of modified lecithin to change the health, growth or life cycle of the plant or plant part.

In one aspect, the present invention relates to a method for improving the quality of a plant part (e.g., the quality of fruits, vegetables, flowers or tubers) by treating the plant part or its corresponding plant with an effective amount of modified lecithin. As an example, the method can be used to improve the turgidity, color and flavor of fruits and vegetables and to reduce fruit cracking. Modified lecithin that can be employed in the methods of the present invention include enzyme-modified lecithin (EML) and chemically modified lecithin such as acetylated lecithin (ACL) and hydroxylated lecithin (HDL).

In another aspect, the present invention relates to a method of retarding senescence in a plant part by treating the plant part or its corresponding plant with an effective amount of modified lecithin. The retardation of senescence can lead to prolonged storage and shelf life for a variety of products such as fruits, vegetables, flowers and tubers.

In another aspect, the present invention relates to a method for increasing the size, weight or both of a plant part (e.g., fruits) by treating the plant part or its corresponding plant with an effective amount of modified lecithin.

In another aspect, the present invention relates to a method for stimulating the growth of a plant or plant part by treating the plant or plant part with an effective amount of modified lecithin. This method can be used to enhance root formation and development of roots on cuttings, to enhance tuber formation, and to stimulate turf grass growth.

In another aspect, the present invention relates to a method of improving the aesthetic attributes of a plant or plant part by treating the plant or plant part with an effective amount of modified lecithin. A plant or plant part with improved aesthetic attributes will look more appealing to an ordinary consumer.

In another aspect, the present invention relates to a method for increasing fruit set on a plant or reducing fruit drop by treating the plant or a suitable part thereof with an effective amount of modified lecithin.

In another aspect, the present invention relates to a method of protecting a plant or plant part from a stress-related injury by treating the plant or plant part with an effective amount of modified lecithin.

In other aspects, the present invention relates to methods of eliciting the hypersensitive response in a plant or plant part, which can be detected by measuring the increase in the total activity of one or more enzymes such as phenylalanine ammonia lyase (PAL), polyphenol oxidase (PPO), peroxidase (POD) and indole-3-acetic acid oxidase (IAA oxidase) in a plant or plant part, and increasing lignin synthesis in a plant or plant part by treating the plant or plant part with an effective amount of modified lecithin.

In another aspect, the present invention relates to a method for protecting a plant or plant part from a stress-related injury caused by an abiotic or biotic stress. The method involves adding an effective amount of modified lecithin into the agrochemical intended to be applied to the plant or plant part.

### BRIEF DESCRIPTION OF THE DRAWINGS

FIG. 1 shows changes in protein content and PAL activity in radish cotyledons exposed to 1-amminocyclopropane-1-carboxylic acid (ACC, a precursor to ethylene), kinetin, and EML all at 20 mg/L.

FIG. 2 shows short-term kinetics of PAL activity in EML-treated radish cotyledons.

FIG. 3 shows effect of EML on lignin content of kinetin-induced expanding cotyledons of radish.

FIG. 4 shows changes in POD activity in cotyledons of radish exposed to ACC, kinetin, or EML.

FIG. 5 shows PAL activity in leaves of mung bean seedlings treated with or without EML (both 20 mg/L) via the transpiration stream.

FIG. 6 shows the effect of LPE and EML on PPO activity in radish cotyledons.

FIG. 7 shows the effect of LPE and EML on IAA oxidase activity in radish cotyledons.

FIG. 8 shows the effect of lecithins on the activity of IAA oxidase in expanding radish cotyledons.

FIG. 9 shows the impact of soy EML on grape firmness.

FIG. 10 shows the impact of soy EML on apple firmness.

FIG. 11 is a product-limit survival fit survival plot, which illustrates the ability of 1000 ppm soy EML aqueous solution to improve vine-ripe tomato fruit storage when applied pre-harvest.

FIGS. 12-14 illustrate the sizing impact of soy EML applied approximately 2 weeks prior to harvest in Fowler, Calif. on Summer Sweet peaches.

FIGS. 15 and 16 illustrate the color impact of soy EML applied approximately 2 weeks prior to harvest in Fowler, Calif. on Summer Sweet peaches.

FIGS. 17-19 illustrate the sizing impact of soy EML, applied approximately 10% color break in Mendota, Calif. on red bell peppers.

FIGS. 20 and 21 illustrate the sizing impact of soy EML applied approximately 3 weeks prior to harvest on McIntosh apples in Gays Mills, Wis.

FIGS. 22-24 illustrate the root formation impact of 20 ppm soy EML solution on mung bean rooting. FIGS. 22 and 23 are pictures of control and EML-treated roots at the end of the experiment. FIG. 24 shows the average number of roots in the control and EML-treated group at the end of the experiment.

FIG. 25 illustrates the impact of soy EML on fruit drop of McIntosh apples conducted in Gays Mills, Wis.

### DETAILED DESCRIPTION OF THE INVENTION

It is disclosed here that modified lecithin, including the relative low cost EML, ACL and HDL, can deliver a variety of beneficial effects when applied to a plant or plant part by changing the health, growth or life cycle of the plant or plant part. The term “life cycle” is used broadly here to encompass both the pre-harvest and post-harvest stages of the plant or plant part. In general, modified lecithin can improve the quality and overall health, stimulate the growth and retard the senescence process in a plant or plant part. The modified lecithin can also increase fruit set, reduce fruit drop and protect a plant or plant part from stress-related injuries. Based on these properties, modified lecithin can be applied in many different ways to benefit the plant industry. For example, modified lecithin can be applied to improve the quality of fruits, vegetables, tubers and cut flowers in terms of their turgidity, color, flavor and scent, and to reduce fruit cracking. Modified lecithin can also be applied to prolong the storage and shelf life of various plant parts such as fruits, vegetables, tubers and cut flowers through retarding or delaying the senescence process in these plant parts. By taking advantage of the growth stimulation activity of modified lecithin, one can increase the size and/or weight of fruits, vegetables and tubers, stimulate turf grass growth, and increase the number of tubers, roots and shoots. One can also make a plant or plant part more appealing to consumers by using modified lecithin to improve the overall health of the plant or plant part. Furthermore, modified lecithin can be applied to increase fruit production by increasing fruit set and reducing fruit drop. In addition, modified lecithin can be used to reduce crop loss caused by stress-related injuries. The beneficial effects disclosed here are applicable to all plants and plant parts that have commercial value (e.g., fruits, flowers, leaves, roots and stems). Preferably, the present invention is practiced on fruits, vegetables, tubers, cut flowers, and their corresponding plants. The present invention is also preferably practiced on turf grass, bedding plants and other functional and decorative plants.

At the physiological level, inventors discovered that EML can trigger a cascade of hypersensitive reactions in a plant that are characterized by the induction of a variety of enzymes, such as lignin synthesizing enzymes including PAL, POD and PPO, leading to the synthesis and deposition of additional lignin to the plant cell walls (see examples below). This response is similar to the self-defense hypersensitive response seen in plants that have been infected by pathogens (e.g., fungi, bacteria or viruses), which secrete one or more elicitors that induce the response. Through the induction of PAL, POD, PPO and other enzymes, the elicitor-induced hypersensitive response is known to impact the direction of carbon flux (e.g., to increase phenylpropanoid, isoprenoid and phytoalexin production) which in turn causes various physiological response such as growth of vegetative and reproductive organs, color development and stress mitigation (Hammond-Kosack K., and Jones J 2000 Responses to Plant Pathogens, In: Biochemistry & Molecular Biology of Plants, Buchanan B B, Gruissem W, and Jones R L eds. American Society of Plant Biologists, Rockville, Md.). One of the end results that relates to stress mitigation is the collapse of the infected plant tissue, which traps and thus prevents the pathogens from infecting other parts of the plant. Without intending to be limited by theory, the inventors believe that the hypersensitive response triggered by EML, which occurs in the absence of a physical wound, is not as dramatic as that triggered by an elicitor from a pathogen and thus does not lead to tissue collapse nor does it impede normal tissue function. However, the limited additional amount of lignin deposited to the cell walls is sufficient to reinforce the cell walls and provide additional structural integrity to plant tissues. As a result, the plant or plant part can better retain water, nutrients and other essential components, leading to better overall quality and health. For harvested plant parts such as fruits, vegetables, tubers and cut flowers, this will also lead to the retardation or delay of the senescence process and thus prolong their storage and shelf life. For living plants and plant parts, this can translate into better growing capabilities, which for example can lead to bigger and heavier products. Furthermore, the improved structural integrity and ability to retain important components can lead to increased fruit set and a reduction in fruit drop. In addition, the plant or plant part can better withstand various stress situations.

As used herein, the term “modified lecithin” means a lecithin modified to enrich its constituency of plant growth modifying compounds, specifically including EML, ACL, HDL and other similar modified lecithins that have plant growth beneficial effects disclosed here for the specific modified lecithins EML, ACL, and HDL. Using the effects noted for EML, ACL and HDL as examples below, one of ordinary skill in the art can test other modified lecithins for the beneficial effects disclosed here and demonstrated in the examples below using the techniques described here. To the extent that the exact efficacy of a particular modified lecithin is not demonstrated in the examples below, it can be easily determined by a skilled artisan through routine experimentation with the systems described in the examples or other systems that a skilled artisan is familiar with. For example, a skilled artisan can use the radish cotyledon system described in Example 1 to measure either lignin deposition or at least one of the PAL, POD, PPO and IAA oxidase enzymatic activities. If a modified lecithin increases lignin deposition or the enzymatic activities measured, the modified lecithin is within the scope of the present invention.

Commercially, lecithin refers to a complex product derived from animal or plant tissues that is commonly used as a wetting and emulsifying agent in a variety of commercial products and is not normally expected to have biological effects in plants. Lecithin contains acetone-insoluble phospholipids (including phosphatidylcholine (PC), phosphatidylethanolamine (PE), phosphatidylinositol (PI), phosphatidic acid (PA), phosphatidylglycerol (PG), phosphatidylserine (PS) and other phospholipids), sugars, glycolipids, and some other substances such as triglycerides, fatty acids, and cholesterol. Refined grades of lecithin may contain any of these components in varying proportions and combinations depending on the type of fractionation used. In its oil-free form, the preponderance of triglycerides and fatty acids is removed and the product contains 90% or more phosphatides representing all or certain fractions of the total phosphatide complex. The consistency of both natural grades and refined grades of lecithin may vary from plastic to fluid, depending upon free fatty acid and oil content, and upon the presence of absence of other diluents. Its color varies from light yellow to brown, depending on the source and on whether it is bleached or not (usually by hydrogen peroxide and benzoyl peroxide). Lecithin is only partially soluble in water, but it readily hydrates to form emulsions. The oil-free phosphatides are soluble in fatty acids, but are practically insoluble in fixed oils. When all phosphatide fractions are present, lecithin is partially soluble in alcohol and practically insoluble in acetone. In a preferred embodiment of the present invention, a food-grade lecithin is used as the starting material to make modified lecithin. This will minimize the safety and environmental concerns over applying modified lecithin to food products. However, a non-food-grade lecithin can also be employed. By current definition, a food-grade lecithin (CAS: 8002-43-5) has the following properties: (1) acetone-insoluble matter (phosphatides) is not less than 50%; (2) acid value is not more than 36; (3) heavy metals (as Pb) is not more than 0.002%; (4) hexane-insoluble matter is not more than 0.3%; (5) lead is not more than 10 mg/kg; (6) peroxide value is not more than 100; and (7) water is not more than 1.5%.

EML refers to a lecithin that has been enzymatically modified (e.g., by phospholipase A₂ or pancreatine), a modification done to enhance the surfactant or emulsifying characteristics of the lecithin. Chemical procedures can also be used to make similar modifications as those made by phospholipase A₂. In a preferred embodiment, a food-grade EML is used in the present invention to minimize the safety and environmental concerns. However, non-food-grade EML can also be employed. By current definition, a food-grade EML has the following properties: (1) acetone-insoluble matter (phosphatides) is not less than 50%; (2) acid value is not more than 40%; (3) lead is not more than 1 ppm as determined by atomic absorption spectroscopy; (4) heavy metals (as Pb) is not more than 20 ppm; (5) hexane-insoluble matter is not more than 0.3%; (6) peroxide value is not more than 20; (7) water is not more than 4%; and (8) lysolecithin is 50 to 80 mole percent of phosphatides as determined by “Determination of Lysolecithin Content of Enzyme-Modified Lecithin: Method 1 (1985),” which is incorporated by reference in its entirety.

Examples of chemically modified lecithin include ACL and HDL. These chemical modifications were also intended to enhance the surfactant or emulsifying characteristics of the lecithin. ACL can be prepared by treating lecithin with acetic anhydride. Acetylation mainly modifies phospholipids into N-acetyl phospholipids. HDL can be prepared by treating lecithin with hydrogen peroxide, benzoyl peroxide, lactic acid and sodium hydroxide, or with hydrogen peroxide, acetic acid and sodium hydroxide, to produce a hydroxylated product having an iodine value preferably 10% lower than that of the starting material. Also preferably, the separated fatty acid fraction of the resultant product has an acetyl value of about 30 to about 38.

EML, ACL and HDL are commonly used as wetting or emulsifying agents and are not normally expected to be biologically active in plants. The inventors demonstrated for the first time that they can deliver a variety of biological effects as described in the examples below. It is noted that the unmodified lecithin does not cause the same effects. It is known in the art that pure lysophospholipids, such as LPE, can cause some of the EML-induced effects disclosed herein. However, the same effects that EML has cannot be explained by the lysophospholipids contained therein. In comparison to pure lysophospholipids, EML is a much more complicated product that contains many other types of molecules, which render EML as a whole, a different product from pure lysophospholipids in terms of its constituents and chemical and physical characters. In the radish cotyledon bioassay described in the examples below, 20 mg/L EML was more effective than 20 mg/L LPE for the induction of hypersensitive response in terms of the activation of enzymes PPO and IAA oxidase, even though the total amount of lysophospholipids in 20 mg/L EML is much less than that in the 20 mg/L LPE. These data indicate that one or more non-lysophospholipid components or chemical/physical properties of EML are important for the effects observed. Furthermore, the fact that ACL and HDL, which are not enriched in lysophospholipids, were also able to induce the activity of IAA oxidase, is consistent with the notion that modified lecithin works differently from pure lysophospholipids.

Lecithin can be obtained from a variety of animal and plant sources including egg yolks, soybeans, sunflowers, peanuts, sesame and canola. The source and process for producing lecithin and methods for enzymatically (e.g., by phospholipase A₂) or chemically modifying lecithin are known to the art. In addition, lecithin, EML, ACL and HDL are commercially available from a variety of sources such as Solae, LLC (Fort Wayne, Ind.). Examples of EML and chemically modified lecithin that can be used in the present invention can be found in Food Chemicals Codex, 4^{th} ed. 1996, pages 198-221; and 21 C.F.R. sec.184.1063, sec. 184.1400 and sec. 172.814, both of which are herein incorporated by reference in their entirety.

In one aspect, the present invention relates to a method of improving the quality of harvested plant parts such as fruits, vegetables, flowers and tubers by treating the plant parts with an effective amount of modified lecithin. In a related aspect, the present invention relates to a method for retarding senescence and enhancing the storage and shelf life of the harvested plant parts by treating the plant parts with an effective amount of modified lecithin. For these applications, modified lecithin can be applied to the plant part either before or after they are harvested. As discussed above, modified lecithin's effects on the quality, senescence and storage and shelf life of a plant part is believed to relate to its ability to reinforce the cell walls and provide additional structural integrity to plant tissues. A harvested plant part is usually limited to the water, nutrients and other essential molecules including its structural components that were there at the time of harvest. Over time, with the loss of these molecules and components, the plant part will undergo the senescence process, leading to the rotting and degradation of the plant part. By reinforcing the cell walls and providing more structural integrity, modified lecithin allows the plant part to better preserve the above molecules and components and thus improve the quality of the plant part. Further, the degradation and senescence process can be retarded as a result and the storage and shelf life of the plant part can be prolonged. For cut flowers wherein the stems are often immersed in water or a nutrient solution of some kind, the quality can still be improved and the shelf life be prolonged by including modified lecithin in the treatment solution.

As used herein, the meaning of “quality of a plant part” depends on the plant part in question and refers to at least one of the following: the firmness (turgidity), color, flavor, scent and cracking of the plant part. The quality of the plant part is considered to be improved if the plant part is firmer (more turgid) and/or has a more desirable color, flavor or scent to an average consumer. For fruits, cracking reduction is also considered an improvement in quality.

In another aspect, the present invention relates to a method for increasing the size, weight or both of a plant part by treating the living plant or the plant part thereof with an effective amount of modified lecithin. The size of a plant part refers to its volume. A skilled artisan knows how to measure and compare the size of a particular plant part. For example, for a substantially round fruit, diameter can be used as a measure of fruit size. For leaves that have similar thickness, the surface area can be used as an indication of leave size. The present invention is particularly useful for increasing the size, weight or both of various fruits, foliage, flowers and tubers. As shown in the examples below, as a result of the size increase, the number of marketable apples from an apple tree was increased.

In a related aspect, the present invention relates to a method of enhancing root formation and development of roots on cuttings by treating the cuttings with an effective amount of modified lecithin. By enhancing root formation or development of roots on cuttings, we mean that modified lecithin can increase the number of roots, the overall length of the roots, or both. When a root is a commercial product itself, the method can be used to increase root production. Otherwise, the method of the present invention can be used to stimulate the growth and development of a plant. In particular, modified lecithin can be added to potting soil media to promote root formation and development.

In another related aspect, the present invention relates to a method for enhancing tuber formation by treating a tuber plant or the tuber thereof with an effective amount of modified lecithin. By enhancing tuber formation, we mean that modified lecithin can increase the number of tubers.

In another related aspect, the present invention relates to a method of stimulating turf grass growth by treating the turf grass with an effective amount of modified lecithin. Turf grass growth can be measured by any method familiar to a skilled artisan. For example, dry weight or biomass of the turf grass can be measured.

In another aspect, the present invention relates to a method of improving the aesthetic attributes of a plant or plant part by treating the plant or plant part with an effective amount of modified lecithin to improve the overall health of the plant or plant part. Without intending to be limited by theory, the inventors believe that modified lecithin achieves this effect by reinforcing the plant cell walls and providing more structural integrity to plant tissues. This activity of modified lecithin is particularly useful in making the turf grass, bedding plants and other functional and decorative plants more appealing to consumers.

In another aspect, the present invention relates to a method of increasing fruit set on or reducing fruit drop from a plant by treating the plant or a suitable part thereof with an effective amount of modified lecithin. Preferably, the whole plant is sprayed with a solution that contains modified lecithin. By increasing fruit set, the number of fruits available for harvest can be increased. By reducing fruit drop, one can reduce fruit loss and potentially increase fruit size as well. The method is particularly useful for fruits such as apples wherein a relatively large number of fruits tend to drop prior to harvest.

In another aspect, the present invention relates to a method for protecting a plant, or plant part from a stress related injury. The method involves applying to the plant or plant part an effective amount of modified lecithin. By protecting a plant or plant part from a stress related injury, we mean one or more of the following: (1) complete prevention of the injury; (2) reduction in severity of the injury; (3) recovery from the injury to a higher degree; and (4) speedier recovery from the injury.

As used herein, the term “stress-related injury” refers to an injury resulting from an abiotic and/or a biotic stress. “Abiotic stress” refers to those non-living substances or environmental factors which can cause one or more injuries to a plant or plant part. Examples of abiotic stress include but are not limited to chilling, freezing, wind, hail, flooding, drought, heat, soil compaction, soil crusting and agricultural chemicals such as pesticides, insecticides, fungicides, herbicides and fertilizers. “Biotic stress” refers to those living substances which cause one or more injuries to a plant or plant part. Examples of biotic stress include but are not limited to pathogens (e.g., fungi, bacteria and viruses), insects, nematodes, snails, mites, weeds, and physical damage caused by human and non-human animals (e.g., grazing, and treading). To protect a plant or plant part from stress-related injuries, modified lecithin can be applied at one or more of the following stages: (1) prior to exposure to stress; (2) during exposure to stress; and (3) after exposure to stress. Furthermore, modified lecithin can be used as an adjuvant for plant growth regulators, pesticides, insecticides, fungicides, herbicides, fertilizers and other agrochemicals that people normally use on plants wherein the use can deliver stress to plants.

In practicing the present invention, a skilled artisan can readily determine whether to apply modified lecithin to only one particular plant part or the whole plant. Using stress-related injury protection as an example, if a stress condition only affects one particular plant part and the goal is to protect that particular part, it may be sufficient to treat that particular plant part with modified lecithin.

Any suitable method of treating a plant or plant part with modified lecithin can be used in the present invention and a skilled artisan is familiar with these methods. Preferably, a plant or plant part is treated with a solution that contains modified lecithin. The preferred solvent for modified lecithin for the purpose of the present invention is water. However, other suitable solvents such as organic solvents can also be used. To treat a plant or plant part with a solution that contains modified lecithin, the plant or plant part can be sprayed with the solution, or it can be dipped or soaked in the solution. Other suitable methods of exposing a plant or plant part to modified lecithin can also be used. For cut-flowers in particular, they can be treated by dipping the cut end of the stem in a modified lecithin-containing solution. For treating underground roots or tubers, modified lecithin can be included in the soil.

The dosage of modified lecithin to be applied for a particular application and the duration of treatment will depend on the type of plant or plant part being treated, the method modified lecithin is being applied, the purpose of the treatment and other factors. A skilled artisan can readily determine the appropriate treatment conditions. Generally speaking, when modified lecithin such as EML is delivered to a target plant or plant part in a solution, its concentration can range from about 1 ppm to about 20,000 ppm, from about 10 ppm to about 10,000 ppm or from about 25 ppm to about 5,000 ppm. The term “about” is used in the specification and claims to cover concentrations that slightly deviate from the recited concentration but retain essential function of the recited concentration.

In addition to modified lecithin, one or more additives that enhance wettability, uptake and effectiveness of modified lecithin can be used together with modified lecithin in practicing the present invention. Examples of additives that can be used in the method of the present invention include but are not limited to ethanol and agricultural adjuvants such as Tactic™ (Loveland Industries, Inc., Greeley, Colo.). The additives can be present in amount of from about 0.005% to about 5% (v/v), from about 0.025% to about 1% (v/v), or from about 0.03% to about 0.5% (v/v) in a treatment composition or formula.

By way of example, but not limitation, examples of the present invention are described below.

### EXAMPLE 1

### Effects of EML on Cotyledon Expansion and Hypersensitive Response Enzymes

Materials and Methods

The soy EML (Precept™ 8160™), ACL (Precept™ 8140™) and HDL (Precept™ 8120™) used in this example were purchased from Solae, LLC (Fort Wayne, Ind.). The egg EML was purchased from Primera Foods, Cameron, Wis.

Seeds of Raphanus sativus L. cv. Cherry-Belle were germinated in darkness at 24° C. for 40 h in Petri dishes containing filter paper wetted with distilled water. The smaller of the two cotyledons was excised, the fresh weight determined, and 10 cotyledons placed adaxial side down on filter paper in Petri dishes containing 7.5 mL of phosphate buffered saline (PBS, 2 mM, pH 6.0) and the compounds to be tested at 20 mg/L. Cotyledons were then incubated under continuous illumination up to 72 h at 24° C. or 25° C. and the increase in fresh weight determined. Chlorophyll content was determined after extraction of tissue into 80% EtOH (containing butylated hydroxytoluene 10 mg/L) and quantified using the equations Chl a=(13.95A663)-(6.88A647) and Chl b=(24.96A652)-(7.32A663) as described by Lichtenthaler, H K (Methods in Enzymology 148:350-382, 1987). IAA oxidase, PAL, PPO and POD activity were determined as described by Kato, M et al. (Plant and Cell Physiology 41:440-447, 2000) and Li, X et al. (Plant Science 164:549-556, 2003).

Results

In order to remove variability from the bioassay—due presumably to temporal changes in the concentration of root-derived cytokinins in cotyledons—the bioassay procedure was modified to routinely include 0.2 mg/L (approximately 1 μM) kinetin in the background.

Cotyledon expansion growth: The effect of soy EML in the presence of kinetin on expansion growth was investigated and the results are shown in Table 1. In the presence of kinetin, soy EML resulted in an increase of cotyledon expansion growth relative to the control.

TABLE 1
Effect of soy EML on kinetin-induced cotyledon expansion in radish.
Ten cotyledons were incubated on filter discs wetted with 2 mM PBS
(pH 6.0) containing either kinetin (20 mg/L) with or without EML (all 20 mg/L).
Cotyledons were incubated under continuous illumination in incubation
chamber at 25° C. for 72 h and the change in fresh weight and chlorophyll
content determined.

| | Change in | | Chlorophyll | Chlorophyll | |
|---|---|---|---|---|---|
| | fresh weight | | a + b | a + b | Chlorophyll |
| Treatment | (mg) | % of control | (μg/cotyledon) | (mg/g FW) | a/b |
| Control | 10.11 ± 1.33 | 100 | 31.57 ± 0.31 | 2.12 | 0.75 |
| ACC | 2.56 ± 0.39 | 25 | 35.90 ± 6.13 | 5.40 | 0.83 |
| Kinetin | 15.49 ± 1.81 | 153 | 54.10 ± 7.03 | 2.17 | 0.87 |
| Kinetin/EML | 18.59 ± 1.13 | 184 | 58.44 ± 5.76 | 2.47 | 0.93 |

In a similar experiment with cucumber cotyledons, the effect of EML on cotyledon expansion growth was tested with both soy and egg EML. As shown in Table 2, both soy and egg EML increased the cotyledon expansion growth.

TABLE 2
Effect of soy and egg EML on expansion growth of cucumber
cotyledons. Cotyledons were incubated on filter discs wetted
with 2 mM PBS buffer (pH 6.0) containing kinetin (0.2 mg/l)
with or without the lecithins (20 mg/L). Cotyledons were
incubated under continuous illumination in an incubation chamber
at 25° C. for 72 h and the change in fresh weight determined
(n = 3).

| Treatment | Change in fresh weight (%) | % of control |
|---|---|---|
| Control | 199.6 ± 1.0 | 100 |
| Soy EML | 232.0 ± 16.6 | 116 |
| Egg EML | 245.4 ± 3.1 | 123 |

In a separate experiment, the effect of EML, ACL and HDL on cotyledon expansion growth were tested. All these modified lecithins increased the cotyledon expansion growth (Table 3).

TABLE 3
Effect of soy EML, ACL, and HDL on expansion growth of radish
cotyledons. Cotyledons were incubated on filter discs wetted with
2 mM PBS buffer (pH 6.0) containing kinetin (0.2 mg/L) with or
without the lecithins (20 mg/L). Cotyledons were incubated under
continuous illumination in an incubation chamber at 25° C. for
72 h and the change in fresh weight and chlorophyll content
determined (n = 3).

| Treatment | Change in fresh weight (mg) | % of control |
|---|---|---|
| Control | 12.60 ± 2.04 | 100 |
| HDL | 14.39 ± 2.09 | 114 |
| ACL | 15.11 ± 2.15 | 120 |
| Soy EML | 14.55 ± 2.69 | 115 |

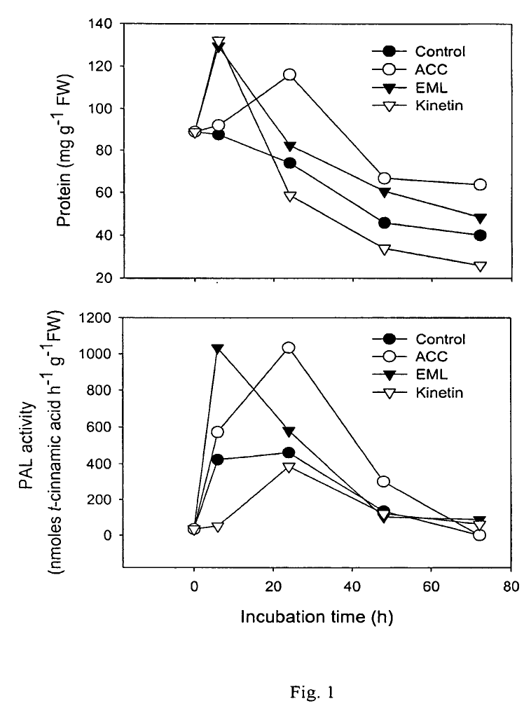

PAL (EC 4.3.1.5) activity: Ethylene is produced by plants in response to a variety of stresses, including wounding (Kato, M et al. Plant and Cell Physiology 41:440-447, 2000). Assuming the stress is of sufficient intensity and duration plants will also begin to show signs of senescence. This notwithstanding, stress is a common daily feature of plant growth and development and because plants are generally immobile they require mechanisms to cope with “normal” day-to-day stress. This is achieved by a system of built-in defense mechanisms. One of these systems involves PAL (EC 4.3.5.1) and activity of this enzyme increases when plants are wounded or exposed to pathogens and/or elicitors. Activity of PAL is also light regulated so transfer of dark-grown seedlings to light would be expected to increase enzyme activity. To determine whether EML acts as an elicitor in a hypersensitive-type response, the activity of PAL in radish cotyledons after exposure to soy EML was investigated and the results are shown in FIG. 1.

EML caused a rapid but transient increase in protein content similar to that observed in kinetin-treated cotyledons. In this treatment, protein content started to decline after 6 h. In ACC-treated cotyledons protein accumulation was delayed and reached a maximum only 24 h after exposure to light. In all cases, accumulation of protein was associated with increased PAL activity.

In EML-treated cotyledons, the increase in PAL was ballistic whereas it was progressively delayed in ACC, control, and kinetin-treated cotyledons. This observation provides strong evidence for a role for EML as an elicitor capable of stimulating PAL.

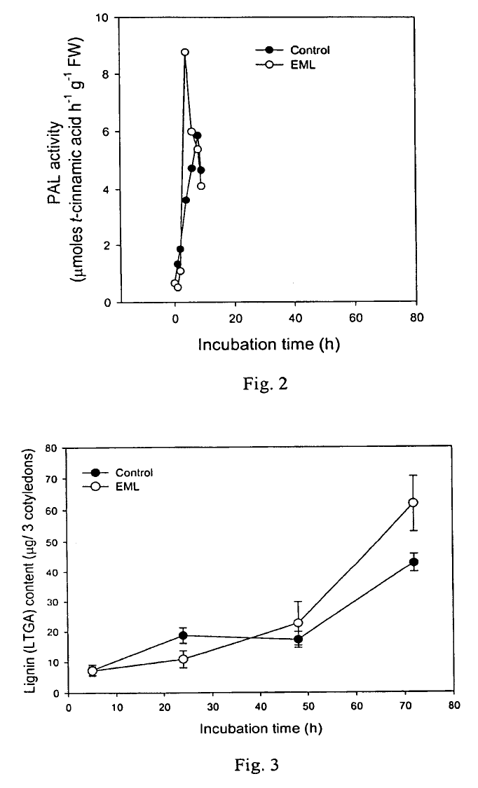

Short-term kinetics of PAL induction by soy EML confirms that PAL activity was increased in EML-treated cotyledons (FIG. 2). Thus, EML activates PAL and likely increases the pheylpropanoid content of growing radish cotyledons. Increased lignin deposition can therefore be expected and lead to the retardation of expansion growth without influencing chlorophyll accumulation. To test this possibility, cotyledons were supplied kinetin (to promote expansion) together with EML and lignin content was determined. Lignin was quantified by measuring the amount of lignothioglycolic acid (LTGA) in extractive-free tissue samples prepared from the cotyledons treated with or without EML as described by Chen, M and McClure, J W (Phytochemistry 53:365-370, 2000). The results in FIG. 3 show that by 72 h EML-treated cotyledons contained substantially more LTGA.

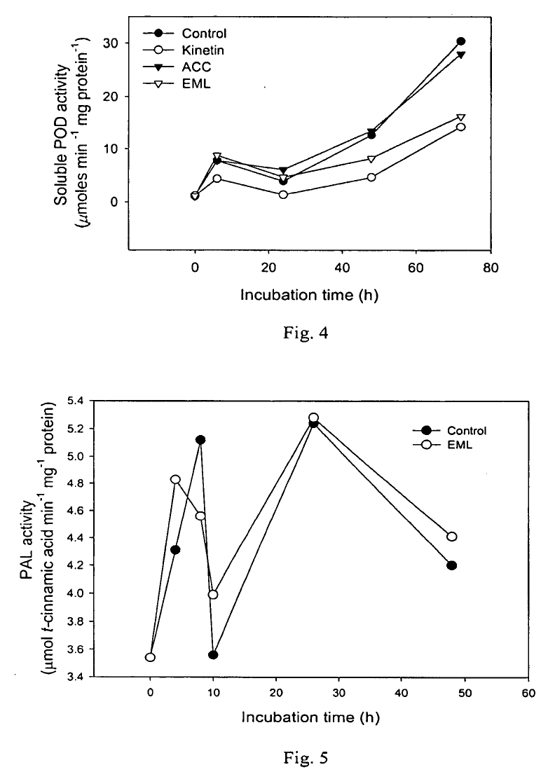

These results, together with induction of PAL (FIGS. 1 & 2) and POD (FIG. 4) activity support the idea that EML acts as an elicitor and causes affected tissues to increase the biosynthesis of phenolic esters and lignin.

POD (EC 1.111.1.7) activity: POD (EC 1.11.1.7) has been implicated in lignin formation at the step of polymerization of monolignols (Grisebach, H, Lignins, In: The Biochemistry of Plants Vol 7, Secondary Plant Products, Conn E E (ed.) Academic Press, New York, pp 457-478, 1981) and induction of POD activity following wounding has been demonstrated for a number of species (Kato, M et al., Plant and Cell Physiology 41:440-447, 2000; and references therein). To determine the effect of EML on induction of POD, activity of this enzyme was monitored during the 72 h incubation period after exposure to soy EML (20 mg/l) and the results are shown in FIG. 4. EML increased POD activity by approximately 15% (relative to control) within the first 6 h of incubation. Thereafter, POD activity declined in all treatments. The increase in POD activity at 48 and 72 h is a normal event in expansion growth and signifies the onset of organ maturity and the commencement of senescence. At this developmental stage, POD activity was lowest in kinetin-treated cotyledons followed by those treated with EML. Highest POD activity was measured in control and ACC-treated cotyledons. This suggests that EML can slow the progression of cotyledon leaf development into the senescence phase.

Although the above result points to induction of components of the hypersensitive response pathway by EML they give no indication of a systemic-type mechanism. To determine whether in fact the response is systemic, mung bean seedlings were supplied solutions of EML via the transpiration stream, incubated for periods up to 72 h, and PAL activity of the cotyledon leaves determined. The results in FIG. 5 show that treatment of mung bean seedlings with EML via the transpiration stream did not change PAL activity in leaves. Thus, we can conclude that EML does not induce a typical systemic-type response.

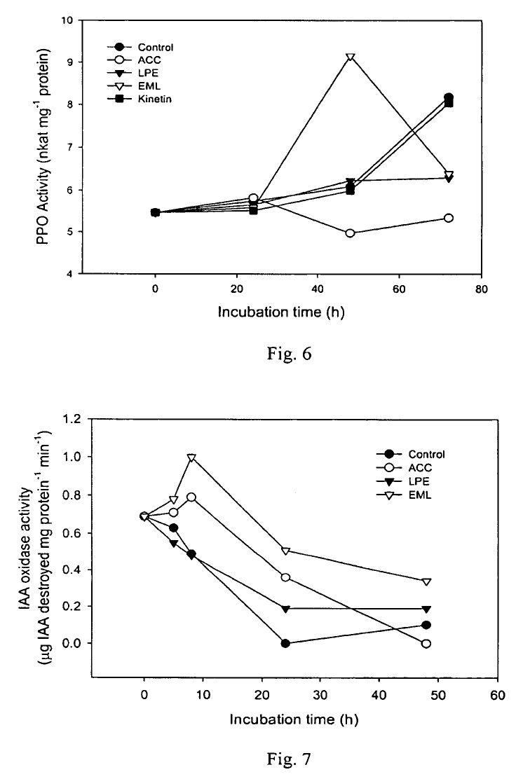

PPO (EC 1.14.18.1): Like PAL and POD, PPO is an important enzyme catalyzing lignin biosynthesis in plants. In the radish system, PAL and POD are induced by exposure to soy EML and as shown in FIG. 6, PPO was also induced and activity was at a maximum 48 h after treatment. By contrast, LPE did not induce PPO activity as EML did and ACC appeared to suppress PPO activity. In untreated and kinetin-treated cotyledons, enzyme activity appeared to increase gradually over time.

IAA Oxidase activity: IAA homeostasis is an important process contributing to correlative control of plant growth and development. Generally, IAA is synthesized in the apices and in shoots; apically derived IAA is basipetally transported. It is the basipetal movement of IAA that modulates process such as apical dominance, adventitious rooting, tropistic responses etc. In the presence of soy EML, activity of IAA oxidase is increased whereas LPE has no apparent effect on this activity (FIG. 7).

POD activity and IAA oxidase are involved in lignin biosynthesis and auxin catabolism respectively. A number of growth retardants have been shown to reduce elongation growth by impacting POD and IAA oxidase activities. In addition, increased IAA oxidase activity has been observed in tissues exposed to pathogens. Thus, the data in FIG. 7 indicates that EML acts as an elicitor and probably contributes to increased phenolic acid production and/or lignification and modulates endogenous IAA by impacting IAA oxidase. To determine whether this effect was due to enzyme modification of the parent lecithin, unmodified (soy lecithin) and modified (EML, ACL and HDL) lecithins were compared.

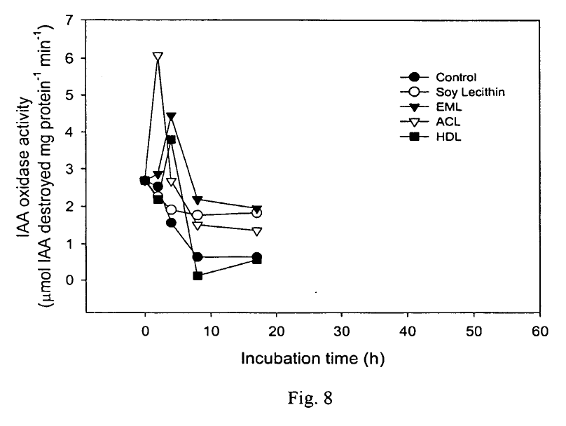

The data in FIG. 8 illustrate that EML, ACL and HDL were very effective inducers of IAA oxidase activity. The unmodified lecithin appeared to have little or no effect on IAA oxidase activity.

### EXAMPLE 2

### Impact of EML on Grape and Apple Firmness (Turgidity)

The EML used in this example was soy EML (Precept™ 8160™) obtained from Solae, LLC (Fort Wayne, Ind.).

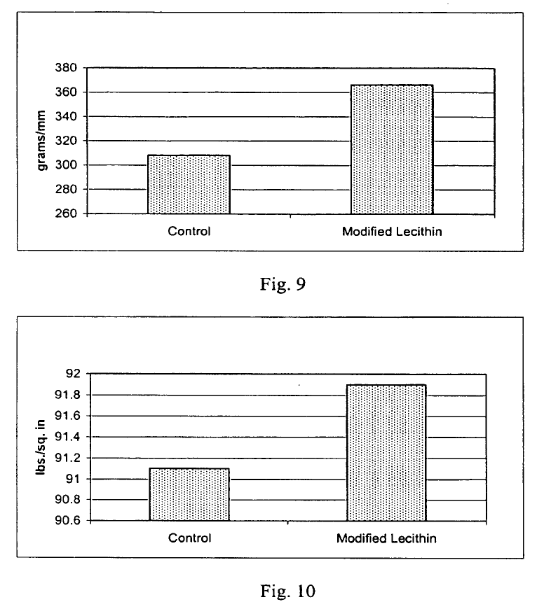

FIG. 9 illustrates the ability of 2000 ppm soy EML aqueous solution to improve grape fruit firmness when applied pre-harvest. Applications of 2000 ppm soy EML were made in April 2003 using a hand operated mist bottle spraying to fully cover the grape clusters with tiny droplets that adhered securely to the fruits without running off. Harvesting took place approximately 2 weeks post application. 25 berries from each cluster were removed from pre-determined sectors of the rachis (with stem cap attached) and measured for firmness using a Firmtech firmness and diameter analyzer (BioWorks, Stillwater, Okla.). As shown in FIG. 9, EML treatment increased the firmness of the grapes.

FIG. 10 illustrates the ability of 2000 ppm soy EML aqueous solution to improve apple fruit firmness when applied pre-harvest. Applications of 2000 ppm soy EML were made on Sep. 18, 2003 with a commercial air blast sprayer to fully cover the apple clusters with tiny droplets that adhered securely to the fruits without running off. Harvesting took place approximately 2 weeks post application. 20 apples were selected at random from the harvested sections and measured for firmness using a Firmtech firmness and diameter analyzer (BioWorks, Stillwater, Okla.). As shown in FIG. 10, EMIL treatment increased the firmness of the apples.

### EXAMPLE 3

### Impact of EML on Tomato Storage Life

The EML used in this example was soy EML (Precept™ 8160™) obtained from Solae, LLC (Fort Wayne, Ind.).

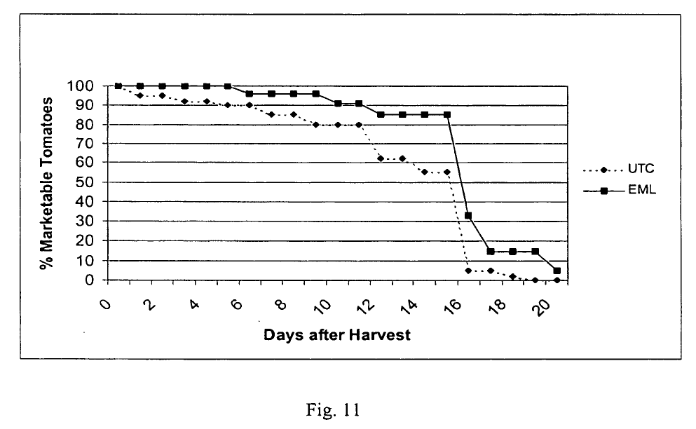

FIG. 11 illustrates the ability of 1000 ppm soy EML aqueous solution to improve vine-ripe tomato fruit storage when applied pre-harvest. Applications of 1000 ppm soy EML were made in July 2003 to mature green tomatoes using a CO₂ backpack sprayer spraying to fully cover the tomato fruit with tiny droplets that adhered securely to the fruits without running off. Harvesting took place approximately 7 days post application. Red ripe fruit remained under light conditions and ambient room temperature for 20 days after harvest with technicians removing unmarketable fruits (fruits showing water-soaking, sour rot, and/or mold). As shown in FIG. 11, EML treatment increased the percentage of total marketable fruit.

### EXAMPLE 4

### Effect of EML on Size, Color and Weight of Fruits and Vegetables

The EML used in this example was soy EML (Precept™ 8160™) obtained from Solae, LLC (Fort Wayne, Ind.).

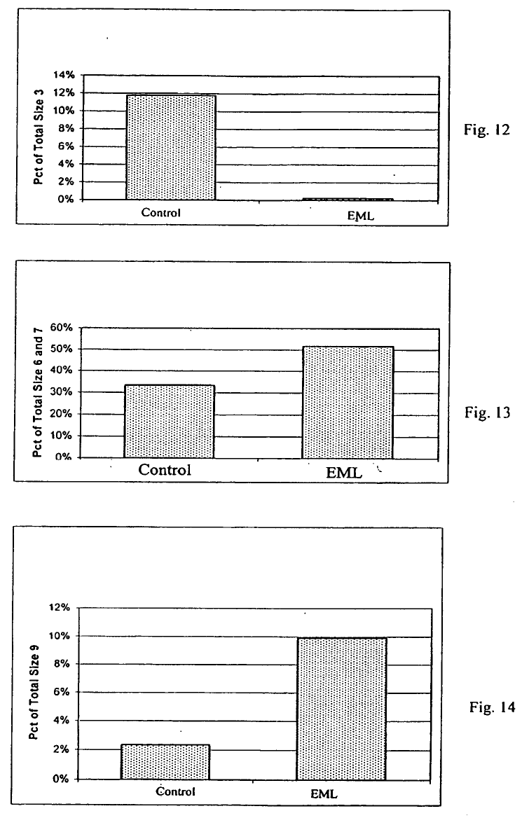

FIGS. 12-16 illustrate the sizing and color impact of soy EML applied approximately 2 weeks prior to harvest in Fowler, Calif. on Summer Sweet peaches. 1000 ppm aqueous solution was applied using a hand operated mist sprayer to fully cover the fruit. Applications took place on Jun. 25, 2003, and harvested on Jul. 8, 2003. Color and size measurements were determined using an optical sorting line at the UC-Davis Kearney Agricultural Station in Fresno, Calif.

This was a Single Latin Square design, with each treatment occupying each available treatment position only once. One scaffold, or limb, was assigned a treatment. All treatments occurred once on each of 4 trees. Treatments were applied in late afternoon. Harvest took place on Jul. 8, 2003. Harvesters stripped all treated fruit from each scaffold and transported them to the Kearney Agricultural Station in Fresno, Calif. Each repetition was run through an optical sorting line to separate fruit by color and size. Sizes range from 1 to 10, with 1 being the smallest most unmarketable fruit approximately 1.5 inches in diameter and 10 being the largest and greater than 3.5 inches in diameter.

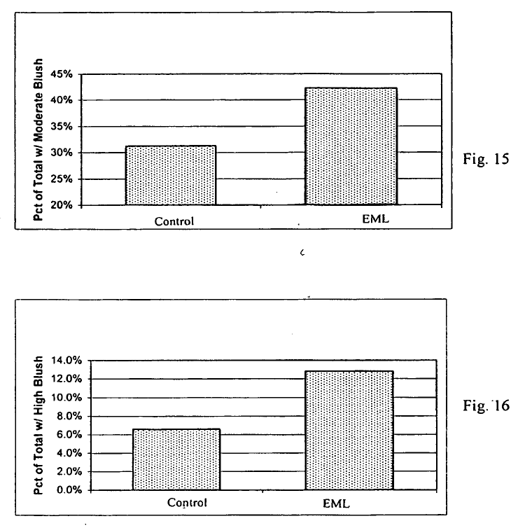

The effect of soy EML on the percentage of size 3, size 6-7 and size 9 peaches are shown in FIGS. 12, 13 and 14, respectively. Treated fruit showed a smaller percentage in the low size category (#3) and much larger percentages in the bigger size categories (#6-9). Larger fruit is more valuable, especially when falling in the moderate to large range of #6-7. Color also determines marketability. Treated fruit show higher percentages of fruit with moderate blush (40-100%) (FIG. 15) surface, and with high blush (60-80%) (FIG. 16).

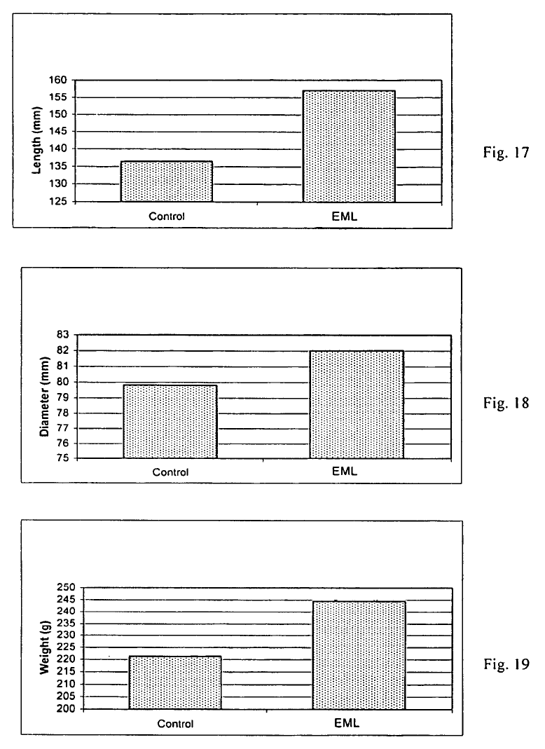

FIGS. 17-19 illustrate the sizing impact of soy EML, applied approximately 10% color break in Mendota, Calif. on red bell peppers on Jul. 23, 2003. 500 ppm aqueous solution was applied using a hand operated mist sprayer to fully cover the fruit. This was a Randomized Complete Block Design with 8 replications. Application took place in the early morning after sunrise. Temperatures were approximately 72° F. and humidity was approximately 50%. Droplet dwell time was in excess of 30 minutes. As can be seen from FIGS. 17-19, treated fruits were longer, wider, and heavier than the control fruits.

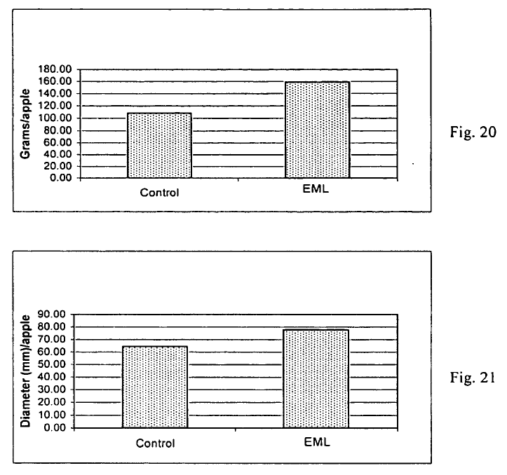

FIGS. 20 and 21 illustrate the weight and sizing impact of soy EML applied approximately 3 weeks prior to harvest on McIntosh apples in Gays Mills, Wis. 1000 ppm aqueous solution was applied using a hand operated mist sprayer to fully cover the fruit. Application took place on Sep. 9, 2003, and harvested Sep. 30, 2003. This was a Single Latin Square design with each treatment occupying only one quadrant in each of 4 tree replicates.

Applications were made in the mid afternoon with an air temperature of approximately 68° F. and clear skies. Droplet dwell time was in excess of 30 minutes. Treated fruit were larger (diameter) and heavier than the control fruit. As illustrated in FIGS. 20 and 21, respectively, soy EML treatment led to an increase in weight and diameter of the McIntosh apples.

### EXAMPLE 5

### EML Enhances Tuber Size and Yield

The EML used in this example was soy EML (Precept™ 8160™) obtained from Solae, LLC (Fort Wayne, Ind.).

To determine the effect of EML on potato tuber size and yield, a field trial was conducted. Dark Red Norland potato plants, grown at Muck Farms, on muck soil, near Lake Mills, Wisconsin, were sprayed with three levels of EML in aqueous solutions. Crop growth at spray application, two weeks before vine kill and four weeks from harvest, was excellent. Tubers were at a stage of rapid accumulation of food stuffs and were rapidly increasing in size.

Field plot design: Uniform part of the field away from the road or other traffic was selected for these experiments. Single row plots, 20 ft long were used. There were five replicates for each treatment and the plots were separated by single untreated rows to avoid any spray drift.

EML levels tested and spray parameters: Three EML levels, namely EML 100 ppm, 250 ppm and 1000 ppm were applied to plant foliage. No adjuvants were used. There were two spray applications. The first application was about two weeks before vine kill where as the 2^{nd} application, 10 days later, was only five days before vine killing.

CO₂ powered backpack sprayer, using nozzle providing fine droplet size, was used. Liquid was applied at of 20 gallons/acre. It enabled a good foliar coverage.

Vine killing: About two weeks before harvest, the plants were sprayed with Paraquat herbicide to kill vines and to prepare for harvest.

Harvest: Central 15 ft of the each plot was manually harvested to determine potato yield. All the tubers were collected, dusted off and weighed. After washing and drying, based on their size, the potatoes w ere classified into <4 oz, 4 to 10 oz and over 10 oz. Each size class was visually further divided, based on their skin color, into premium, acceptable and poor. Potatoes in each class were counted and weighed. Any rotting or damaged potatoes were then discarded.

As shown in Table 4, all three EML levels tested increased potato tuber yield. EML 100 ppm provided the largest marketable yield increase of 36.8%.

As shown in Table 5, all three EML levels tested increased potato tuber size. EML 100 ppm provided the largest increase.

TABLE 4
EML application to the foliage of potato plants of cultivar
Dark Red Norland enhances tuber yield.

| | Marketable tuber yield | |
|---|---|---|
| Treatment | (Lbs/plot) | % of untreated control |
| Untreated Control | 17.0 | 100% |
| EML 100 ppm | 23.3 | 136.8% |
| EML 250 ppm | 18.9 | 110.3% |
| EML 1000 ppm | 21.6 | 127.0% |

TABLE 5
EML application to the foliage of potato cultivar dark
Red Norland enhances tuber size.

| | Tubers <4 oz. | Tubers >4 oz. |
|---|---|---|
| | (expressed as % | (expressed as % |
| Treatment | of total yield) | of total yield) |
| Untreated Control | 32.8% | 67.2% |
| EML 100 ppm | 24.2% | 75.8% |
| EML 250 ppm | 27.2% | 72.8% |
| EML1000 ppm | 25.2% | 74.8% |

### EXAMPLE 6

### EML Enhanced Root Mass

The EML used in this example was soy EML (Precept™ 8160™) obtained from Solae, LLC (Fort Wayne, Ind.).

This example illustrates the ability of EML to promote root growth when incorporated with the sod substrate prior to placement in a hydroponic situation. On Jul. 12, 2003, 3 repetitions of cross-sectional slices measuring 6 inches by 12 inches from a sod mat were placed on a bed of powdered soy EML to coat the root mass. The mats were then placed in a hydroponic solution of ½ strength Hoagland's solution with aeration for 14 days. After 14 days, the mats were removed from solution and three 1-inch slices removed from the mid-section of each mat. The soil was washed from the roots and the shoot portion was sheared at the root shoot interface as to leave only the root portion behind. The root masses were air-dried and then weights taken. The results were shown in Table 6.

In Table 6, each replication consists of three 1-inch by 6-inch cross-section slices of sod from a 6-inch by 12-inch mat in Hydroponic solution. Each replication number is the mean of the raw data root mass in grams of 6 square inches of sod. In all three replications, EML treatment increased the sod root mass.

TABLE 6
Sod root mass in grams.

| | Water Control | Soy EML |
|---|---|---|
| Replication 1 | 2.08 g | 3.54 g |
| Replication 2 | 2.10 g | 3.08 g |
| Replication 3 | 2.45 g | 2.65 g |
| Mean | 2.21 g | 3.09 g |

### EXAMPLE 7

### Effect of EML on Root Formation

The EML used in this example was soy EML (Precept™ 8160™) obtained from Solae, LLC (Fort Wayne, Ind.).

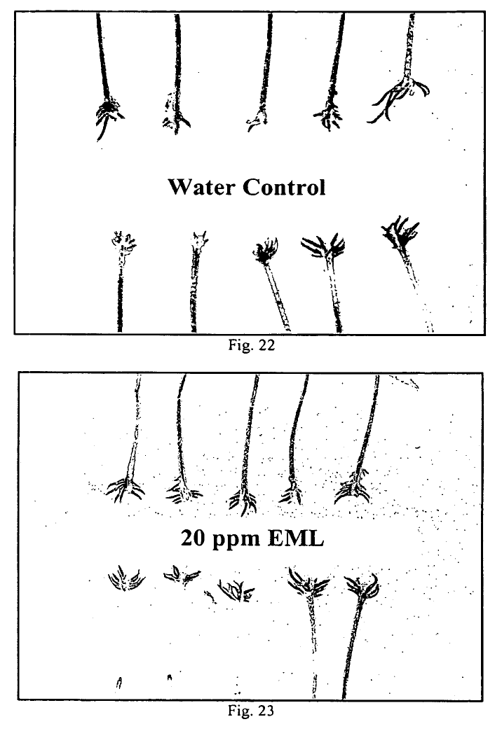

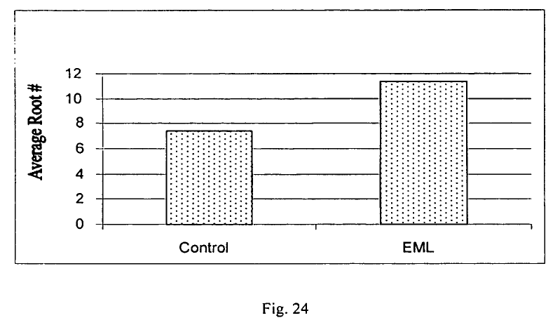

FIGS. 22-24 illustrate the impact of 20 ppm soy EML solution on mung bean root formation. 3.5 cm cuttings were placed in 6-inch test tubes containing solution for 4 days under constant light and approximately 70° F. After 4 days the newly formed roots were counted. Ten replicates were executed. FIGS. 22 and 23 are pictures of control and EML-treated roots at the end of the experiment. FIG. 24 shows the average number of roots in the control and EML-treated group at the end of the experiment. Treated mung bean cuttings showed approximately 50% increase in root number after 4 days of treatment (FIG. 24).

### EXAMPLE 8

### EML Enhanced Pod Set and Seed Yield in Soybean

The EML used in this example was soy EML (Precept™ 8160™) obtained from Solae, LLC (Fort Wayne, Ind.).

In soybeans (Glycine max L), 43 to 81% of flowers produced fail to produce mature pods due to flower drop, before pollination, or fertilized, immature pod drop (Hansen and Shibles, Agronomy Journal. Vol. 70, January-February, 1978). Over the years, various growth hormones such as ABA, IAA, BAP and GA3 have been tested to enhance the pod set with various levels of success (Mosjidis et al., Annals of Botany 71:193-199, 1993).

To determine the effect of EML on soybean pod set and seed yield, ten field trials were conducted with Glycine max L. soybean. Of these, two were large plot farmer's field trials and all others were small plot replicated field tests. Several different cultivars were used. Test sites had diverse growing conditions, ranging from Brownsville, Tex. to Cedar Falls, Iowa, covering the soybean belt as well as the areas where soybeans are grown only on a small acreage.

In field tests, in Brownville, Tex., the plants were sprayed with various levels of EML, in aqueous solutions, at pre-flowering, early and peak flowering stages of plant development. In the subsequent field tests, based on these data, a single spray at peak flowering of plant growth was applied.

Field plot design: In all field tests, wherever possible, uniform part of the field was selected for the experiments. Four row plots, 25 to 30 ft long were used. There were three to five replicates for each treatment. To avoid EML drift to the adjoining plots, only the center two rows were treated and used to record all subsequent data. At farmers field tests, plot size varied from 2 to 8 acres.

EML levels tested and spray parameters: EML levels of 0, 10, 50, 100 and 500 ppm were applied to plant foliage. No adjuvants were used.

CO₂ powered backpack sprayer, using nozzle providing fine droplet size, was used. Liquid was applied at 15 to 50 gallons/acre. It enabled a good foliar coverage.

Pod set data: Pod set data were recorded on ten plants, selected at random, in each replicate about four weeks after the EML spray. All the growing pods on each of the selected plants were counted.

Seed yield data: For seed yield data, the two center rows, treated with EML, were harvested using a combine harvester. Data were calculated based on plot size and compared to the untreated controls.

In all ten field trials, soy EML was effective in increasing the pod set of soybeans. Depending on the specific cultivars, the concentrations of EML that were effective varied somewhat. As an example, the results from a trial conducted in Cedar Falls, Iowa are shown in Table 7 and Table 8. As shown in Table 7, the percentage increase in pod set was higher for cultivar Pioneer 92B38 than cultivar Kruger K-269. All concentrations of EML tested increased the pod set of cultivar Pioneer 92B3 8. For cultivar Kruger K-269, 10 ppm, 50 ppm and 100 ppm EML increased the pod set while 500 ppm EML did not.

As shown in Table 8, with the exception of 10 ppm EML on cultivar Pioneer 92B38, all concentrations of EML tested increased the seed yield of cultivars Pioneer 92B38 and Kruger K-269.

TABLE 7
Soybean field test in Cedar Falls, IA: EML increased pod set of
Pioneer92B38 and KrugerK-269 Cultivars.

| | | | % of | % of |
|---|---|---|---|---|
| | Mean # of | Mean # of | Control | Control |
| | Pods/Plant | Pods/Plant | Pioneer | Kruger |
| Treatment | Pioneer 92B38 | Kruger K-269 | 92B38 | K-269 |
| Untreated | 16.5 | 27.0 | 100% | 100% |
| EML 10 ppm | 22.5 | 28.0 | 136% | 104% |
| EML 50 ppm | 27.5 | 31.5 | 167% | 117% |
| EML 100 ppm | 23.5 | 30.0 | 142% | 111% |
| EML 500 ppm | 26.0 | 26.0 | 158% | 96% |

TABLE 8
Soybean field test in Cedar Falls, IA: EML increased
soybean yield of cultivars Pioneer 92B38 and Kruger K-269.

| | Yield (Bushels/Acre) | | Yield (Bushels/Acre) | |
|---|---|---|---|---|
| Treatment | Pioneer | Kruger | Pioneer | Kruger |
| Untreated Control | 32.88 | 23.78 | 100% | 100% |
| EML 10 ppm | 32.78 | 25.24 | 100% | 106% |
| EML 50 ppm | 35.18 | 27.04 | 107% | 114% |
| EML 100 ppm | 35.58 | 25.14 | 108% | 106% |
| EML 500 ppm | 33.50 | 25.64 | 102% | 108% |

### EXAMPLE 9

### Effect of EML on Fruit Drop

The EML used in this example was soy EML (Precept™ 8160™) obtained from Solae, LLC (Fort Wayne, Ind.).

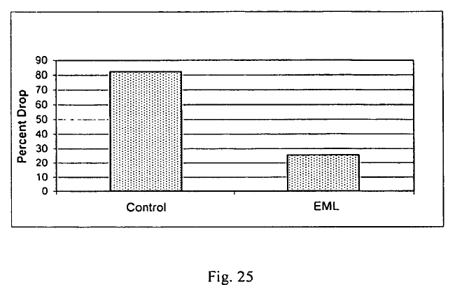

FIG. 25 illustrates the impact of soy EML on fruit drop when applied approximately 3 weeks prior to harvest on McIntosh apples in Gays Mills, Wis. 1000 ppm soy EML aqueous solution was applied using a hand operated mist sprayer to fully cover the fruit. Application took place on Sep. 9, 2003, and harvested Sep. 30, 2003. This was a Single Latin Square design with each treatment occupying only one quadrant in each of 4 tree replicates.

Applications were made in the mid afternoon with an air temperature of approximately 68° F. and clear skies. Droplet dwell time was in excess of 30 minutes. McIntosh apple trees often drop a large portion of their fruit. As shown in FIG. 25, treated fruit showed a much lower fruit drop rate.

### EXAMPLE 10

### Protecting Plants from Stress-Related Injuries

Materials and Methods

The experiments were conducted in growth rooms located at the University of Wisconsin Biotron Facility (2115 Observatory Drive, Madison, Wis. 53706). Each growth room was 10 ft×10 ft where temperature, light quality and photoperiod were controlled. The lights were at about 8 feet above the floor. A solid bank of fluorescent tubes provides lighting, while humidification was provided by steam pipes injected into the intake vents approximately 1 foot below the ceiling on the walls adjacent to the door. The outflow ducts were located directly below the intake vents approximately 1 foot off of the floor. Within these growth rooms the plants were grown on benches approximately 3.5 feet off the floor.

All plants mentioned were grown in 6-inch square plastic (HDPE) pots approximately 6 inches deep with one of several soil-less media as indicated in each individual experiment, unless otherwise noted. The seeds were planted four per pot, uniformly in each corner of the pot into Fafard's Super Fine Germinating Mix soil-less media (Fafard Corp., 1471 Amity Road, Anderson, S.C. 29621). Once planted the pots were placed in a growth room set at 80% relative humidity (RH), 25° C. ±2° C., 16 hour photoperiod and 400 uE of light at the top of the canopy.

Soy EML (Precept™ 8160™) was purchased from Solae, LLC (Fort Wayne, Ind.). EML-containing solutions were prepared by mixing EML in water with aggressive agitation until EML was completely dissolved or suspended. Solutions containing specific concentrations of EML as indicated in Tables 9-12 were used to treat plants as described below.

Soy EML was used to make solutions that were applied directly to the vegetative parts of growing plants. To simulate the calcium found in normal tap water, all EML-containing solutions contained 1 mM of CaCl₂. In some cases, 0.032% Tactic™ (Loveland Industries, Inc., Greeley, Colo.), a combination of an organo-silicone and a synthetic latex, and in others, ethanol, was further added to the EML-containing solution to facilitate wetting of the plant surface by the solution. The solution was applied to the plants by spraying with a hand held, manual spray bottle, similar to those used to dispense household cleaners.

Results

Chilling Stress Alleviation in Field Corn with a Pre-stress Application of EML: Four seeds of Golden Harvest field corn (F-1 hybrid, H-2387) were planted in six-inch square plastic (HDPE) pots. Fourteen days after planting, all the four plants in each pot were sprayed with 500 ppm of EML solution without any adjuvants or with water, which served as control. For each replicate, pots with plants matching in growth and development were selected. To ensure statistical validity, control and treatment were assigned to pots, at random. After spray, the plants were allowed to sit under ambient conditions for six hours before being exposed to the cold stress. Cold stress was initiated at the beginning of night period by dropping the temperature to 0C and the day temperature warmed to 25° C. This day/night temperature (25/0° C.) was repeated for four days. At the end of four cycles the plants were returned to their original growing conditions (25/21° C., day/night temperature) and allowed to grow for an additional five days to determine the effect of the cold on growth and vigor. After five days of growth, the plants were harvested at the soil level with a scalpel and fresh weight of each treatment was taken and compared against that of the control pot. In this experiment, using 500 ppm EML, we observed an increase in fresh weight of 5.3% over the control. This would indicate a mitigation, or alleviation, of the cold stress that would allow the treated plants to resume normal growth rates more quickly.

Treatment of Soybean Plants with EML to Alleviate Cold Stress: In this experiment, soybean cultivar KB 241(Kaltenberg Seed Farms, 5506 State Road 19, PO Box 278, Waunakee, Wis. 53597) was used. The soybeans were planted in the six-inch pots, as described earlier, but eight plants per pot, two per corner, uniformly spaced with respect to the four corners. The plants were grown in Scott's 366-P soil-less growing media (Scott's Corp., 14111 Scottslawn Road, Marysville, Ohio 43041) under conditions: 80% RH, 25° C. and 400 uE of light for a fourteen-hour photoperiod in a growth room. Six days after planting the plants were treated with EML in the manner as described above in “Chilling Stress Alleviation in Field Corn with a Pre-stress Application of EML.” In addition to the EML and CaCl₂, Tactic, a common spray adjuvant, was added at 0.032% to improve wettability of the leaf surface by the spray solution. In this experiment, one half of each pot, four plants, were treated with a control spray and the other four with treatment (EML 500 ppm). Plants in two halves of pots were matched for size, growth and development. The assignment of the treatment and control was at random. Consistent with the previous experiment, the application was made six hours prior to the cold exposure, after which the pots were moved to a growth room under cold (0° C.) conditions for 72 hour. The RH was at 80% and 400 uE of light for a 14-hour photoperiod. At the end of three days the plants were returned to their original growing conditions at 25° C. ±2° C., 80% RH and 400 uE of light and harvested after 13 d growth. Harvest was consistent with that described in “Chilling Stress Alleviation in Field Corn with a Pre-stress Application of EML”: cutting the vegetative portion of the plant at the soil surface with a scalpel and measuring the fresh weight of the plants. In this experiment EML treatment prior to chilling stress led to a fresh weight increase of 22% over the water treated, paired control. This increase is indicative of mitigated stress during the cold period and increased vigor after the stress.

Treatment of Field Corn Plants to Mitigate Drought Stress: Golden Harvest field corn (F1 hybrid, H-2387), planted in six-inch square plastic (HDPE) pots was used. The seeds were planted four per pot, uniformly in each corner of the pot into Scott's 366-P soil-less growing media (Scott's Corp. 14111 Scottslawn Road, Marysville, Ohio 43041). The plants were grown in a greenhouse for twenty days at normal growing conditions (27° C. ±2° C. daytime for 14 hours and 23° C. ±2° C. nighttime). Humidity was not controlled and six 600 W high pressure sodium lights approximately 4.5 feet above the growing benches were placed to provide supplemental light. These greenhouses are located at the University of Wisconsin Biotron (2115 Observatory Drive, Madison, Wis. 53706). After 20 days of plant growth in pots, drought stress was initiated by withholding water to the pots until two days after visual symptoms of wilting appeared. At this time, each pot was divided into two side-by-side sets of two plants, one side was treated with EML and the other side was treated with water (control). Pots were fully watered to release the stress on plants and were kept under good water conditions for 9 days. Plants were then harvested and fresh weight recorded. As shown in Table 9, 100 ppm and 500 ppm EML treatment following drought stress led to a fresh weight increase of 6.1% and 10.3% respectively over the water treated, paired control.

TABLE 9
Fresh weight of corn plants treated with EML to mitigate
the drought stress. Data are average of five replicates.

| Treatment | Average Mass/Plant (g) |
|---|---|
| EML (100 ppm) | 32.68 |
| Paired water control for the 100 ppm-EML | 30.68 |
| group | |
| EML (500 ppm) | 33.61 |
| Paired water control for the 500 ppm-EML | 30.48 |
| group | |

Mid-Stress Application of EML to Mitigate Drought Stress on Corn Plants: Golden Harvest field corn (F1 hybrid, H-2387) planted in six-inch square plastic (HDPE) pots was used. The seeds were planted four per pot, uniformly in each corner of the pot into Scott's 366-P soil-less growing media (see details in “Treatment of Field Corn Plants to Mitigate Drought Stress” above). All the details in this experiment are the same as described above in “Treatment of Field Corn Plants to Mitigate Drought Stress” except that EML spray application was made at one day after visual wilting was seen as opposed to two days after wilting in “Treatment of Field Corn Plants to Mitigate Drought Stress.” Plants were harvested seven days after the release of water stress. As shown in Table 10, 500 ppm EML treatment following drought stress led to a fresh weight increase of 19.5% over the water-treated, paired control.

TABLE 10
Fresh weight of corn plants treated with EML to mitigate
the drought stress. Data are average of five replicates.

| Treatment | Mean plant mass (g) |
|---|---|
| EML500 ppm | 25.07 |
| Water Control | 20.98 |

Mid- and Late-Stress Application of EML to Mitigate Drought Stress in Corn: The experiments above in “Treatment of Field Corn Plants to Mitigate Drought Stress” were repeated with Golden Harvest and Syngenta N60-N2 field corn plants. Details of the experiments and the stress conditions were the same.

Twenty-one day old Golden Harvest and Syngenta N60-N2 field corn plants were treated with 500 ppm EML during and just before the end of the drought stress. Mid-stress application took place after one day of drought stress measured from the time when plants first showed the signs of wilting. The late-stress application took place after 2 days of drought stress measured from the time when plants first showed the signs of wilting. The plants were watered within one hour of the last treatment application. The experiment had four replicates for each treatment. Eight days after stress relief, the plants were harvested and data were collected.

As shown in Table 11, EML application increased biomass in both Golden Harvest and Syngenta N60-N2 corn. This increase was more pronounced in Syngenta N60-N2 corn plants. Application at either mid- or late-drought period was effective.

TABLE 11
The effect of EML application in mid- (one day after drought stress)
and late-drought (two days after drought stress, which was just
before stress relief) stress periods on fresh weight of Golden
Harvest and Syngenta N60-N2 corn plants.

| | % increase in fresh weight |
|---|---|
| | over control by 500 ppm |
| | EML |
| Mid-drought application on Golden | 13.0% |
| Harvest corn plants | |
| Late-drought application on Golden | 10.9% |
| Harvest corn plants | |
| Mid-drought application on | 28.9% |
| Syngenta N60-N2 corn plants | |
| Late-drought application on | 22.2% |
| Syngenta N60-N2 corn plants | |

Pre-stress Application of EML to Mitigate Cold Stress in Cucumbers: Fifteen-day-old Dasher variety cucumbers were treated with 500 ppm EML and 1000 ppm EML before exposing plants to cold stress. Plants were in 6-inch square plastic (HDPE) pots with 2 plants in a pot placed diagonally from each other in opposite corners of the pot. Both plants in the pot were sprayed with the same treatment. There were 6 replicates for each treatment. Plants were sprayed with treatment or water, allowed to dry and then placed in a 1-2° C. cold room in the University of Wisconsin Biotron (room 251B) for 14 to 16 hours. After cold treatment, plants were allowed to grow in normal temperature conditions for 8 days. Plants were then harvested and data were collected. A treatment of cucumber plants with EML at 500 ppm and 1000 ppm before chilling stress gave 3.5% and 16.3% increase in fresh weight respectively compared to water treated control plants.

Post-stress Application of EML to Mitigate Cold Stress in Cucumbers: Experiment in “Pre-stress Application of EML to Mitigate Cold Stress in Cucumbers” was repeated except that the application of EML, was made after the cold stress and cold treatment was for a 24-hour period.

Twenty-two day old Dasher cucumber plants were cold stressed by placing them in a 1-2° C. cold room in the University of Wisconsin Biotron (room 251B) for a 24 hour period. Immediately after removal from the cold room, the plants were sprayed with treatment or water control. Twenty days after treatment, plants were harvested and data were collected. At harvest time, the degree of damage and re-growth varied widely. However, EML treatment (500 ppm) gave 90.3% increase in biomass as compared to water treated control plants.

Pre- and Post-stress Application of EML to Mitigate Cold Stress in Melons: Experiments in “Pre-stress Application of EML to Mitigate Cold Stress in Cucumbers” and “Post-stress Application of EML to Mitigate Cold Stress in Cucumbers” were repeated with melons.

Thirteen-day-old Primo melons were treated with 500 ppm EML before or after being exposed to cold stress. At the time of treatment, the plants had one fully expanded leaf and one small leaf. The plants were sprayed with treatment solutions either prior to cold stress or right after cold stress. Cold stress was exposure of plants to 1-2° C. for a 12-hour period. Plants were in 6-inch square HDPE pots with 2 plants in a pot placed diagonally from each other in opposite corners of the pot. Both plants in the pot were sprayed with the same treatment. There were 3 replicates for each treatment. Eight days after treatment, plants were harvested and data were collected. At time of harvest, the degree of damage and re-growth varied widely. At the time of harvest, all of the old leaves showed very little to no damage, all plants had 2-3 new leaves, all seem to be healthy and growing from apical meristem, and flower buds were beginning to form on all plants. EML at 500 ppm was effective at recovery from stress when applications were made after the cold stress exposure (Table 12).

TABLE 12
The effect of EML application before and after cold stress on
fresh weight of Primo melons.

| | % increase in fresh weight |
|---|---|
| | over control by 500 ppm |
| | EML |
| EML treatment before cold | 9.4% |
| stress | |
| EML treatment after cold stress | 11.4% |

Mitigation of Cold Stress in Tomato Plants: Experiments described in “Pre-stress Application of EML to Mitigate Cold Stress in Cucumbers” and “Post-stress Application of EML to Mitigate Cold Stress in Cucumbers” were repeated with tomatoes.

Fifty-two-day old Florida 47 tomatoes were treated with 500 ppm EML or 1000 ppm EML before exposure to cold stress. At the time of treatment, the plants were about 42-48 cm tall. The plants were arranged in replicates: replicate I being the most advanced (at flowering stage) and the tallest and replicate 4 being the least advanced and shortest. Replicates 2 and 3 were in-between. There were paired four replications for water control. After spraying, the plants were allowed to dry and then put into a 1-2° C. cold room for 25 hours. Plants were left in the normal growing conditions for several days after the cold stress. At the time of harvest, the plants were about 55-65 cm tall. The lower (old growth) leaves were all very damaged and many had fallen off but all plants had significant new growth. EML applied at 500 ppm and 1000 ppm gave 4.4% and 12.7% increase in plant biomass over control, respectively.

Although the invention has been described in connection with specific examples, it is understood that the invention is not limited to such specific examples but encompasses all such modifications and variations apparent to a skilled artisan that fall within the scope of the appended claims.

We claim:

1. A method for improving the quality of a plant part selected from a fruit, a flower, a leave, and a stem, the method comprising the step of treating the plant part or the corresponding plant with a composition that comprises modified lecithin in an amount sufficient to improve the quality of the plant part.

2. The method of claim 1, wherein the quality of the plant part comprises at least one of the characters selected from turgidity, color, flavor and fruit cracking.

3. The method of claim 1, wherein the modified lecithin is enzyme-modified lecithin.

4. The method of claim 1, wherein the plant part is a fruit.

5. The method of claim 1, wherein the stem is tuber.

6. The method of claim 1, wherein the plant part is exposed to the composition before it is harvested from the plant.

7. The method of claim 1, wherein the plant part is exposed to the composition after it is harvested from the plant.

8. A method for retarding senescence in a plant part comprising the step of treating the plant part or the corresponding plant with a composition that comprises modified lecithin in an amount sufficient to retard senescence in the plant part.

9. The method of claim 8, wherein senescence retardation is measured by storage or shelf life extension of the plant part.

10. The method of claim 8, wherein the modified lecithin is enzyme-modified lecithin.

11. The method of claim 8, wherein the plant part is a fruit.

12. The method of claim 8, wherein the plant part is a tuber.

13. The method of claim 8, wherein the plant part is exposed to the composition before it is harvested from the plant.

14. The method of claim 8, wherein the plant part is exposed to the composition after it is harvested from the plant.

15. A method for increasing the size, weight or both of a plant part selected from a fruit, a flower, a leave, and a stem, the method comprising the step of treating the plant part or corresponding plant with a composition that comprises modified lecithin in an amount effective to increase the size, weight or both of the plant part.

16. The method of claim 15, wherein the modified lecithin is enzyme-modified lecithin.

17. The method of claim 15, wherein the plant part is a fruit.

18. The method of claim 15, wherein the stem is tuber.

19. A method for stimulating the growth of a plant part selected from a fruit, a flower, a leave, and a stem, the method comprising the step of treating the plant part or the corresponding plant with a composition that comprises modified lecithin in an amount sufficient to stimulate the growth of the plant or plant part.

20. The method of claim 19, wherein the composition is used to enhance tuber formation by treating a tuber plant or tubers thereof with the composition.

21. The method of claim 19, wherein the modified lecithin is enzyme-modified lecithin.

22. The method of claim 19, wherein the plant part is a fruit.

23. The method of claim 19, wherein the stem is tuber.

24. A method for increasing fruit set on a plant comprising the step of treating the plant or a suitable part thereof with a composition that comprises modified lecithin in an amount effective to increase fruit set on the plant.

25. The method of claim 24, wherein the modified lecithin is enzyme-modified lecithin.

26. A method for reducing fruit drop from a plant comprising the step of treating the plant or a suitable part thereof with a composition that comprises modified lecithin in amount effective to reduce fruit drop.

27. The method of claim 26, wherein the fruit is apple.

28. The method of claim 26, wherein the modified lecithin is enzyme-modified lecithin.

29. A method for protecting a plant or plant part from a stress-related injury comprising the step of treating the plant or plant part with a composition that comprises modified lecithin in an amount effective to protect the plant or plant part from a stress-related injury.

30. The method of claim 29, wherein the modified lecithin is enzyme-modified lecithin.

31. The method of claim 29, wherein the stress injury is the result of an abiotic stress.

32. The method of claim 31, wherein the abiotic stress is the result of chilling, freezing, wind, hail, flooding, drought, heat, soil compaction, soil crusting, agricultural chemical, or a combination of at least two of the foregoing.

33. The method of claim 29, wherein the stress injury is the result of a biotic stress.

34. The method of claim 33, wherein the biotic stress is caused by a pathogen, an insect, a nematode, a snail, mites, weeds, or a physical damage caused by human and non-human animals.

35. A method for improving the aesthetic attributes of a plant or plant part comprising the step of treating the plant or plant part with a composition that comprises modified lecithin in an amount effective to improve the aesthetic attributes of the plant or plant part.
